(19) United States
(12) Patent Application Publication
(10) Pub. No.: US 2006/0165775 A1
(43) Pub. Date: Jul. 27, 2006

(54) Capsule and film-forming composition comprising gum arabic

(75) Inventors: Yuri V. Korshak, Moscow (RU); Abdulakh K. Mikitaev, Moscow (RU)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: eastgate Investments Limited

(21) Appl. No.: 10/532,384

(22) PCT Filed: Oct. 22, 2002
(86) PCT No.: PCT/RU02/00459

§ 371 Date: Apr. 21, 2005

(51) Int. Cl. A61K 9/48

(52) U.S. Cl. 424/451

(57) ABSTRACT

A capsule for oral delivery of a composition comprises 60% to 95% by weight of gum arabic and to the remainder a water-soluble polymer, a hydrocolloid and a plasticizer. The proportion of gum arabic is preferably 70% to 90% by weight of all components. The water-soluble polymer is preferably a hydroxypropylmethylcellulose or carboxymethylcellulose or alginetes. The hydrocolloid is preferably a carrageenan or agar gum or galactomannan or a mixture thereof. The capsule may be is containing medicinal or pharmaceutical agents as fill material. The capsule has good mechanical strength and low brittleness. The presence of gum arabic in a solution raises the solubility of other film-forming polymers.

### FIELD OF THE INVENTION

The present invention relates to capsules for oral delivery, which contain medicinal, pharmaceutical or other agents as fill materials. Concurrently, the invention relates to compositions used for preparation of such capsules.

### BACKGROUND OF THE INVENTION

Soft capsules are widely used in the food and pharmaceutical industries for encapsulating vitamins, medications, cosmetics, paint, pigment and other substances in the form of emulsions or oil solutions. The applications of soft capsules have markedly increased due to the growing consumption of products in a convenient, encapsulated form.

Hard capsules are generally used in medicine as containers for medications, allowing their successive delivery into the digestive system. Shell formation is typically accomplished by immersing shaping pins in an aqueous solution of gelatine containing the required ingredients, and heating the solution. Hard capsules used for the oral delivery of medications should have the ability to dissolve in aqueous media. They may also be required to disintegrate under the influence of intestinal media over a required period.

Gelatine is commonly used for preparing both soft and hard capsules. Gelatine can be obtained from pig and cow by-products such as bones, skins and white connective tissue, and has good mechanical and processing properties. In spite of the wide application of gelatine capsules in medicine, large groups of people are not able to ingest them, for ethnic or religious reasons. In addition, recently observed cross-species contamination from cattle with bovine spongiform encephalopathy (BSE) to humans has had a negative impact on the use of gelatine capsules.

As an alternative to gelatine-based capsules, capsules for medical use can be made from various water-soluble synthetic polymers, capable of forming strong and elastic films. For example, WO 97/35537 A1 International Application Publication discloses the use of polyvinyl alcohol, polyethylene oxide and polycaprolactone in soft-shell capsule preparation. This is achieved by elastically deforming two films to a desired shape, followed by filling and sealing the film about the filling material. However, the process requires the films to be solvated with appropriate solvent prior to encapsulation to cause partial solvation of the material surface, such that surface can adhere to and seal the film material.

WO 00/27367 A1 International Publication discloses the use of water-soluble cellulose derivatives as film-forming materials for soft capsule shell preparation. One such material is hydroxypropylmethylcellulose (HPMC). In this case, capsules generally comprise a film 18 to 200 μm in width, laminated on one side with natural gums such as carrageenan, gum arabic or soya bean proteins. An intermediate adhesive layer is applied between the two layers. However, these capsules are brittle, and the films lose their original transparency. This has been attributed to the use of low molecular weight HPMC.

WO 98/27151 A1 International Publication describes soft capsules produced using water-soluble cellulose ethers in combination with hydrocolloids and cross-linking agents. Cellulose ethers with alkyl or hydroxyalkyl pendant groups, such as methyl, hydroxyethyl, hydroxypropyl and hydroxyethylmethylcellulose can be used. The preferred cellulose ether is HPMC, which is recommended as a basic material (up to 95%) in composition for soft capsules, is used in the form of 2% solutions. Other components include hydrocolloids, plasticizers and sequestering agents. However, the use of HPMC as a basic film-forming material is limited due to low solubility of this substance in water; a large amount of water is required in the film production process.

U.S. Pat. No. 6,214,376 B1 patent, which is the most similar to the present invention, discloses the compositions for soft and hard capsules for oral delivery, as well as the composition of capsules themselves. The water solution of composition according to U.S. Pat. No. 6,214,376 B1 comprising a plasticizer, kappa-carrageenan, and at least one non-termoreversible gum is selected from group consisting of hydrolyzed starches, dexrins, proteins, polyviniylpyrrolidone and gum arabic. Those additional materials may be present at levels from 0% to about 25% or more of the composition.

At the same time the carrageenan comprises at least 50% by weight of all film-forming material in the above composition. The carrageenan therefore may be present as 75% or 50% by weight of all gums in composition. However, carrageenan, like other hydrocolloids, is not capable of forming highly concentrated solutions and thus has little use in this technology.

Chemically-modified natural materials can be used for the preparation of hard gelatine-free capsules. Capsules comprising water-soluble cellulose ethers or cellulose ethers with polyvinyl acetate have been proposed. Shape-forming is achieved by immersing pins of the appropriate size into an aqueous solution, followed by drying. However, the use of these materials causes slipping of the capsules from the pins and the formation of a wrinkled surface. Another disadvantage of capsules made from cellulose ethers is their tendency to form cracks during removal from the pins. Processing also requires additional heating of the pins making the use of standard equipment impossible.

The various conventional gelatine-free capsules described above, may also be contaminated with harmful chemical reagents. This is particularly undesirable for capsules for use in medicine or the food industry.

Gum arabic is produced by natural exudation of the acacia tree (Acacia Senegal). It is a brittle, tough solid material comprising a mixture of amber-coloured, partially transparent pieces of various size and shape. Gum arabic is a natural bio-polymer with a molecular weight up to 9.0-10⁵ g/mol and a polysaccharide chemical structure comprising about 13% of carboxyl groups, 0.3% of nitrogen and 4% of inorganic salts. The chemical structure of the polysaccharide fraction is complicated and includes residues of galactose, arabinose, rhamnose and glucuronic acid in amounts of 45÷46%, 23→24%, 13÷14% and 14÷16%, respectively, with the ratio of 3/2/1/1. This material is relatively cheap compared to other natural gums and does not contain tannins. Studies have shown that casting gum arabic from an aqueous solution produces films of low mechanical strength and extreme brittleness and thus the material has not been used as a major component in capsule preparations.

### SUMMARY OF THE INVENTION

The present invention is based on the surprising discovery that the presence of gum arabic in a solution raises the solubility of other film-forming polymers. Thus, it has been discovered that a solution of gum arabic and other film-forming polymers can be used to produce soft and hard capsules of high mechanical strength and improved flexibility.

According to the main aspect of the invention, a capsule comprises 60% to 95% by weight gum arabic and to the remainder a water-soluble polymer, a hydrocolloid and a plasticizer. The proportion of gum arabic is preferably 70% to 90% by weight. These capsules may be containing medicinal or pharmaceutical agents as fill material.

The water-soluble polymer is preferably alginetes or cellulose ether (e.g. alkyl- and/or hydroxyalkyl-substituted cellulose ether) or hydroxypropylmethylcellulose or carboxymethylcellulose. The hydrocolloid is preferable a carrageenan (kappa-carrageenan) or agar gum or galactomannan or a mixture thereof.

The plasticizer is preferable 1, 2-propylene glycol or glycerol or glycerol triacetate or glucose or sorbitol or sucrose or fructose or maltose or cellobiose or lactose or CaCl₂.7H₂O or triethyl citrate or tributyl citrate or dioctyl sodium sulfosuccinate or polyethylene glycol or carbamide or a mixture thereof

According to the other main aspect of the invention, a film-forming composition for oral delivery capsules comprising in an aqueous solution 60% to 95% by all film-forming components weight of gum arabic and to the remainder a water-soluble polymer, a hydrocolloid and a plasticizer.

A capsule of the invention may be used in drug delivery as having good mechanical strength and low brittleness. At the same time the capsule of the invention comprises an inexpensive major component and therefore can be mass-produced.

### BRIEF DESCRIPTION OF THE DRAWINGS

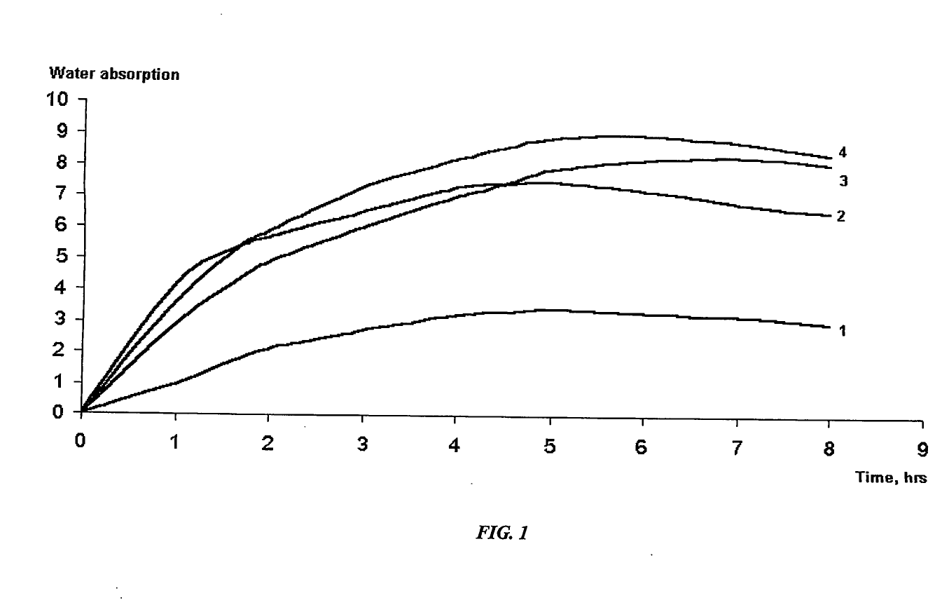

The invention are further explained through description of the preferred embodiments of accomplishment and applied drawing containing the FIG. 1 which is a graph illustrating the kinetic of water absorption from saturated vapor phase at 24° C. by various film-forming materials: Gum Arabic—the Curve 1; hydroxypropilmethyl cellulose—the Curve 2; sodium alginete—the Curve 3; kappa-karageenan—the Curve 4.

### DETAILED DESCRIPTION OF THE PREFERRED EMBODIMENTS

A capsule of the invention comprises at least 60% by weight of gum arabic. It appears that the high proportion of gum arabic that can be used in the invention may be caused by the decrease in the rate of water absorption at high ambient humidity, compared to the values for certain other components that can be used in the invention. Thus, the water-absorption kinetics (at 24° C.) of gum arabic (Curve 1 according to FIG. 1), hydroxypropylmethyl cellulose (Curve 2), sodium alginete (Curve 3) and kappa-carrageenan (Curve 4) are shown in FIG. 1.

A film-forming composition for oral delivery capsules comprise in an aqueous solution 60% to 95% by all film-forming components dry weight of gum arabic and to the remainder a water-soluble polymer, a hydrocolloid and a plasticizer. The proportion of gum arabic is preferably 70% to 90% by weight of all film-forming components dry weight in an aqueous solution.

The water-soluble polymer is cellulose ether, for example alkyl- and/or hydroxyalkyl-substituted cellulose ether, or a hydroxypropylmethylcellulose or carboxymethylcellulose or alginetes. The hydrocolloid includes a carrageenan (kappa-carrageenan) or agar gum or galactomannan or a mixture thereof.

The plasticizer is preferable 1, 2-propylene glycol or glycerol or glycerol triacetate or glucose or sorbitol or sucrose or fructose or maltose or cellobiose or lactose or CaCl₂.7H₂O or triethyl citrate or tributyl citrate or dioctyl sodium sulfosuccinate or polyethylene glycol or carbamide or a mixture thereof

The solubilities of HPMC, CMC and kappa-carrageenan in water at 20° C. are respectively 2, 0.5 and 0.5 g/100 ml. It will be evident from Examples 11 to 16 that higher solubilities (respectively 5.8% HPMC, 6.5% CMC, 15% CMC, 8% HPMC, 13.2% CMC and 9.2% kappa-carrageenan) are observed, in the presence of gum arabic.

Capsules of the invention may be prepared by dissolving gum arabic, preferably in deionised water, to obtain a solution of 1% to 40% by weight gum arabic, dissolving various polymeric components (e.g. starch, polyvinyl alcohol, hydroxypropylmethyl cellulose, carboxymethyl cellulose, carrageenan, alginetes, chitosan etc.) in the solution to a desired concentration, and further dissolving a desired amount of any of plasticizers, sequestering agents and/or mono- or divalent metal salts. The final concentration of polymeric components in solution may be in the range of 1% to 40% by weight.

Films prepared by casting the aqueous solution on to a smooth surface showed a tensile strength value of 50 to 70 kg/cm² and an elongation at break of 120% to 280%. These results demonstrate the applicability of those materials for soft capsule preparation.

Hard capsule shells may be prepared by conventional method, e.g. by immersing shaping pins into the aqueous solution and forming shells around the pins. Mechanical testing of those materials prepared on the basis of gum arabic showed tensile strength values of more than 300 kg/cm² and reasonable rigidity, acceptable for hard capsule shells. Suitable water-soluble cellulose ethers include alkyl- and/or hydroxyalkyl-substituted cellulose ethers, in which there are preferably of 1 to 4 carbon atoms in the alkyl chains. Preferred compounds include methylcellulose, hydroxyethylcellulose, hydroxypropylcellulose, hydroxyethylmethylcellulose and hydroxyethylethylcellulose. Hydroxypropylmethylcellulose (HPMC) is particularly preferred. The amount of cellulose ether or a mixture thereof may be 1% to 35% by weight, preferably 5% to 25% by weight.

The water-soluble polymer may alternatively be a polysaccharide containing carboxyl groups. Suitable such polymers include carboxymethyl cellulose (CMC), carboxymethyl starch and alginetes of alkaline metals, e.g. Li⁺, Na⁺, K⁺ (from seaweeds). These may be used for a gastro-resistant capsule preparation. The amount of carboxylic polysaccharide derivative or a mixture thereof is preferably from 5% to 25% by weight.

Suitable water-soluble synthetic polymers include polyvinyl alcohol, partially hydrolysed polyvinyl acetate, polyvinylpyrrolidone, polyethylene oxide, polypropylene oxide, polyacrylic and polymethacrylic acid, polyacryl amide, halide salts of quaternised polyvinyl pyridine, poly-N, N-dialkyldiallylammonium halides, and similar hydrophilic polymers. Preferred polymers include polyvinyl alcohol, polyethylene oxide and polyacryl amide. The amount of this polymer component is preferably from 1% to 30% by weight.

Suitable water-soluble polysaccharides include starch, dextrins, pectins and chitosan. The preferred polysaccharides include sucrose, starch, alginetes and chitosan. The preferred amount of this component is 0.5% to 30% by weight. Suitable hydrocolloids include gellan gum, carrageenan, agar gum (from seaweed), guar gum, xanthan, galactomannan, funoran, acetan and wellan. Such gums may provide a synergistic effect under the mixing. The preferred gums for this purpose are carrageenan (a mixture of ammonium, magnesium and sodium salts of galactose and 3, 6-anhydrogalactose esters), gellan gum, mannan gum (galactomannan gum/glucomannan gum) and agar. The preferred amount is 0.1% to 10% by weight.

The novel composition includes also a plasticizer such as 1,2-propylene glycol, glycerol, mono-, di- or triacetates of glycerol, sorbitol, sucrose, fructose, maltose, cellobiose, lactose, triethyl citrate, tributyl citrate, dioctyl sodium sulfosuccinate, polyethylene glycols or the like as well as mixtures thereof The amount is preferably from 2% to 40% by weight.

Suitable sequestering agents are ethylenediaminetetraacetic acid (EDTA), boric acid, citric acid, gluconic acid, lactic acid, tartaric acid, phosphoric acid or salts thereof, lecitin, dihydroxyethylglycine and combinations thereof. The preferred amount is preferably 0.01% to 3% by weight, and more preferably 0.1% to 2% by weight.

Monovalent or divalent cations, such as Li⁺, Na⁺, K⁺, NH₄⁺, Ca²⁺ or Mg²⁺ may be used to adjust the extent of sequestering. Such cations will typically be provided in the form of salts.

Capsules of the invention may comprise inert substances such as carbon black, titanium oxide or gypsum. Flavourings, aromatic agents and/or antioxidants may be added as necessary or desired, in order to provide desired mechanical and other properties. A capsule of the invention may also comprise a pharmaceutically or food-acceptable colouring agent, of which suitable examples are riboflavin, carotenes, chlorophyllin, indigocarmin, anthocyanines, caramel and betanin.

The following Examples illustrate the invention. In each Example, the amounts of components are given in percentage by weight.

Examples 1 to 10 illustrate the preparation of soft capsules. The procedure comprised dissolving gum arabic in deionised water, typically at room temperature, to obtain a solution containing 1% to 40% by weight of gum arabic (Solution 1). Other polymeric components such as starch, polyvinyl alcohol, HPMC, CMC, carrageenan, alginetes, chitosan, etc. were then dissolved in the Solution 1, typically at room temperature, to obtain a solution (Solution 2) having the required concentration of various components. Plasticizers, sequestering agents and mono- or divalent metal salts were then dissolved, typically at room temperature, in the Solution 2. The final concentration of polymeric components in the solution may be in the range of 1% to 40% by weight.

Examples 11 to 17 illustrate the preparation of hard capsules. These were prepared by conventional methods, involving immersing shaping pins into the aqueous solution and forming shells around the pins.

### EXAMPLE 1

A composition of 77% gum arabic, 15.3% glycerol, 3.85% CaCl₂.7H₂O and 3.85% carbamide was cast as an aqueous solution comprising 40% gum arabic, to produce an elastic film having a tensile strength of 61 kg/cm² and an elongation at break of 150%.

### EXAMPLE 2

A composition of 70% gum arabic, 20% glycerol, 7% dextrins and 3% starch was cast as an aqueous solution comprising 30% gum arabic, to produce an elastic film having a tensile strength of 51 kg/cm² and an elongation at break of 180%.

### EXAMPLE 3

A composition of 68% gum arabic, 15.8% glycerol, 10% NaCl, 5% glucose and 1.2% polyvinyl alcohol was cast as an aqueous solution comprising 30% gum arabic, to produce an elastic film having a tensile strength of 65 kg/cm² and an elongation at break of 160%.

### EXAMPLE 4

A composition of 75% gum arabic, 19.3% glycerol diacetate, 4.1% CaCl₂.7H₂O and 1.6% kappa-carrageenan was cast as an aqueous solution comprising 40% gum arabic, to produce an elastic film having a tensile strength of 72 kg/cm² and an elongation at break of 120%.

### EXAMPLE 5

A composition of 70% gum arabic, 17% glycerol monoacetate, 8% sorbitol and 5% HPMC was cast as an aqueous solution comprising 35% gum arabic, to produce an elastic film having a tensile strength of 68 kg/cm² and an elongation at break of 170%.

### EXAMPLE 6

A composition of 75% gum arabic, 15% 1,2-propylene glycol, 5% carboxymethyl cellulose and 5% CaCl₂.7H₂O was cast as an aqueous solution comprising 36% gum arabic, to produce an elastic film having a tensile strength of 55 kg/cm² and an elongation at break of 200%.

### EXAMPLE 7

A composition of 71% gum arabic, 18% glycerol, 5% sorbitol, 5% sodium alginete and 1% kappa-carrageenan was cast as an aqueous solution comprising 32% gum arabic, to produce an elastic film having a tensile strength of 70 kg/cm² and an elongation at break of 120%.

### EXAMPLE 8

A composition of 68.5% gum arabic, 20% glycerol, 5% chitosan, 5% carbamide, 0.5% kappa-carrageenan and 1% NaCl was cast as an aqueous solution comprising 40% gum arabic, to produce an elastic film having a tensile strength of 60 kg/cm² and an elongation at break of 130%.

### EXAMPLE 9

A composition of 80% gum arabic, 14% glycerol diacetate, 0.5% kappa-carrageenan, 5% HPMC and 0.5% CaCl₂.7H₂O was cast as an aqueous solution comprising 40% gum arabic, to produce an elastic film having a tensile strength of 61 kg/cm² and an elongation at break of 155%.

### EXAMPLE 10

A composition of 73% gum arabic, 20% dioctyl sodium sulfosuccinate, 5% sorbitol, 1% kappa-carrageenan and 1% EDTA was cast as an aqueous solution comprising 40% gum arabic, to produce an elastic film having a tensile strength of 70 kg/cm² and an elongation at break of 255%.

### EXAMPLE 11

A composition of 70% gum arabic, 12% glycerol, 3% sucrose, 13.5% HPMC and 1.5% kappa-carrageenan was cast into an aqueous solution comprising 30% gum arabic, to produce an elastic film having a tensile strength of 280 kg/cm² and an elongation at break of 12%.

### EXAMPLE 12

A composition of 70% gum arabic, 10% glycerol monoacetate, 4% sucrose, 13% CMC, 2.5% kappa-carrageenan and 0.5% CaCl₂.7H₂O was cast as an aqueous solution comprising 35% gum arabic, to produce an elastic film having a tensile strength of 300 kg/cm² and an elongation at break of 8%.

### EXAMPLE 13

A composition of 61% gum arabic, 6.5% glycerol diacetate, 30% CMC, 2% kappa-carrageenan and 0.5% CaCl₂.7H₂O was cast as an aqueous solution comprising 30% gum arabic, to produce an elastic film having a tensile strength of 400 kg/cm² and an elongation at break of 3%.

### EXAMPLE 14

A composition of 63% gum arabic, 10% glycerol monoacetate, 5% sorbitol, 20% HPMC, 1% kappa-carrageenan and 1% EDTA was cast as an aqueous solution comprising 25% gum arabic, to produce an elastic film having a tensile strength of 510 kg/cm² and an elongation at break of 4%.

### EXAMPLE 15

A composition of 65% gum arabic, 24.5% carboxymethyl cellulose (CMC), 10% glycerol and 0.5% sorbitol was cast as an aqueous solution comprising 35% gum arabic, to produce an elastic film having a tensile strength of 310 kg/cm² and an elongation at break of 5%.

### EXAMPLE 16

A composition of 65% of gum arabic, 20% of kappa-carrageenan, 10% of glycerol monoacetate, 1.5% of citric acid, 0.1% of KCl and 3.4% of sorbitol was cast as an aqueous solution comprising 30% gum arabic, to produce an elastic film having a tensile strength of 410 kg/cm² and an elongation at break of 4%.

### EXAMPLE 17

A composition of 70% gum arabic, 20% sodium alginete, 5% dioctyl sodium sulfosuccinate, 0.5% kappa-carrageenan, 4% sorbitol and 0.5% EDTA was cast as an aqueous solution comprising 35% gum arabic, to produce an elastic film having a tensile strength of 390 kg/cm² and an elongation at break of 6%.

### INDUSTRIAL APPLICATION

A capsule of the invention may be used in drug delivery. As well as having good mechanical strength and low brittleness, a capsule of the invention comprises an inexpensive major component and can be mass produced. Capsules of the invention have a wide range of applications in the food industry, pharmaceutical industry and in the applied medicine.

1. A capsule for oral delivery of a composition comprising 60% to 95% by weight of gum arabic and to the remainder a water-soluble polymer, a hydrocolloid and a plasticizer.

2. The capsule according to claim 1, which comprises 70% to 90% by weight of gum arabic.

3. The capsule according to claim 1, wherein the water-soluble polymer is cellulose ether.

4. The capsule according to claim 1, wherein the water-soluble polymer is an alkyl- and/or hydroxyalkyl-substituted cellulose ether.

5. The capsule according to claim 1, wherein the water-soluble polymer is a hydroxypropylmethylcellulose or carboxymethylcellulose or alginetes.

6. The capsule according to claim 1, wherein the hydrocolloid is a carrageenan or agar gum or galactomannan or a mixture thereof.

7. The capsule according to claim 1, wherein the hydrocolloid is a kappa-carrageenan.

8. The capsule according to claim 1, wherein the plasticizer is 1, 2-propylene glycol or glycerol or glycerol triacetate or glucose or sorbitol or sucrose or fructose or maltose or cellobiose or lactose or CaCl₂.7H₂O or triethyl citrate or tributyl citrate or dioctyl sodium sulfosuccinate or polyethylene glycol or carbamide or a mixture thereof.

9. The capsule according to claim 1, containing medicinal or pharmaceutical agents as fill material.

10. A film-forming composition for oral delivery capsules comprising in an aqueous solution 60% to 95% by all film-forming components dry weight of gum arabic and to the remainder a water-soluble polymer, a hydrocolloid and a plasticizer.

11. The composition according to claim 10, which comprises 70 to 90% by all film-forming components dry weight of gum arabic.

12. The composition according to claim 10, wherein the water-soluble polymer is cellulose ether.

13. The composition according to claim 10, wherein the water-soluble polymer is an alkyl- and/or hydroxyalkyl-substituted cellulose ether.

14. The composition according to claim 10, wherein the water-soluble polymer is a hydroxypropylmethylcellulose or carboxymethylcellulose or alginetes.

15. The composition according to claim 10, wherein the hydrocolloid is a carrageenan or agar gum or galactomannan or a mixture thereof.

16. The composition according to claim 10, wherein the hydrocolloid is a kappa-carrageenan.

17. The composition according to claim 10, wherein the plasticizer is 1,2-propylene glycol or glycerol or glycerol triacetate or glucose or sorbitol or sucrose or fructose or maltose or cellobiose or lactose or CaCl₂.7H₂O or triethyl citrate or tributyl citrate or dioctyl sodium sulfosuccinate or polyethylene glycol or carbamide or a mixture thereof.
